(19) United States
(12) Patent Application Publication
(10) Pub. No.: US 2012/0184773 A1
(43) Pub. Date: Jul. 19, 2012

(54) REDUCTION OF FUSED BICYCLIC IMPURITIES IN TRIIODINATED X-RAY CONTRAST MEDIA

(75) Inventors: Tino J. Caviggiola, III, Hazelwood, MO (US); Michelle M. Jones, O'Fallon, MO (US)

(73) Assignee: MALLINCKRODT LLC, Hazelwood, MO (US)

(21) Appl. No.: 13/497,320

(22) PCT Filed: Sep. 28, 2010
(86) PCT No.: PCT/US2010/050458

§ 371 Date: Mar. 21, 2012

Related U.S. Application Data
(60) Provisional application No. 61/246,997, filed on Sep. 30, 2009.

(51) Int. Cl. C07C 231/12

(52) U.S. Cl. 564/153

(57) ABSTRACT

The present disclosure generally relates to an improved process for alkylating a triiodo-substituted arylamide to form a compound suitable for use as an X-ray contrast agent. More particularly, the present disclosure is directed to such a process that limits the formation of fused bicyclic impurities, such as Impurity G, in the alkylation reaction mixture.

### TECHNICAL FIELD

The present disclosure generally relates to an improved process for alkylating a triiodo-substituted arylamide to form a compound suitable for use as an X-ray contrast agent. More particularly, the present disclosure is directed to such a process that limits the formation of fused bicyclic impurities, such as Impurity G, in the alkylation reaction mixture.

### BACKGROUND OF THE DISCLOSURE

Diagnostic imaging is an important non-invasive tool for the evaluation of pathology and physiology. More particularly, X-ray imaging is a well known and extremely valuable tool for the early detection and diagnosis of various disease states in the human body. The use of contrast agents and/or media for image enhancement in medical X-ray imaging procedures is widespread. A detailed background on contrast agents and media in medical imaging is provided, for example, by D. P. Swanson et al., Pharmaceuticals in Medical Imaging (1990, MacMillan Publishing Company).

Briefly, in X-ray imaging, transmitted radiation is used to produce a radiograph based upon overall tissue attenuation characteristics. X-rays pass through various tissues and are attenuated by scattering, i.e., reflection or refraction or energy absorption. However, certain body organs, vessels and anatomical sites exhibit so little absorption of X-ray radiation that radiographs of these body portions are difficult to obtain. To overcome this problem, radiologists routinely introduce an X-ray absorbing medium containing a contrast agent into such body organs, vessels and anatomical sites.

The production methods commonly used to prepare triiodinated X-ray contrast media or agents typically result in the formation of impurities or byproducts, and/or the presence of unreacted starting components, in the reaction mixture that are difficult to remove. (See, e.g., U.S. Pat. Nos. 5,648,536; 5,204,005; and, 4,396,598; the entire contents of which are incorporated herein by reference for all relevant and consistent purposes.) The presence of these impurities creates a challenge for the manufacturer, at least in part because specifications for such X-ray contrast media or agents impose very low limits on the acceptable amount of such impurities. For example, one impurity that may be encountered in the preparation of iodixanol is the difficult to remove impurity known as “Impurity G”. As illustrated in Scheme 1 below, this impurity is formed by cyclization of the hydroxyl group on the alkylating linker, present between the two molecules (or monomers) that form the dimerized iodixanol compound, with one of the central aromatic rings, with concomitant loss of iodide.

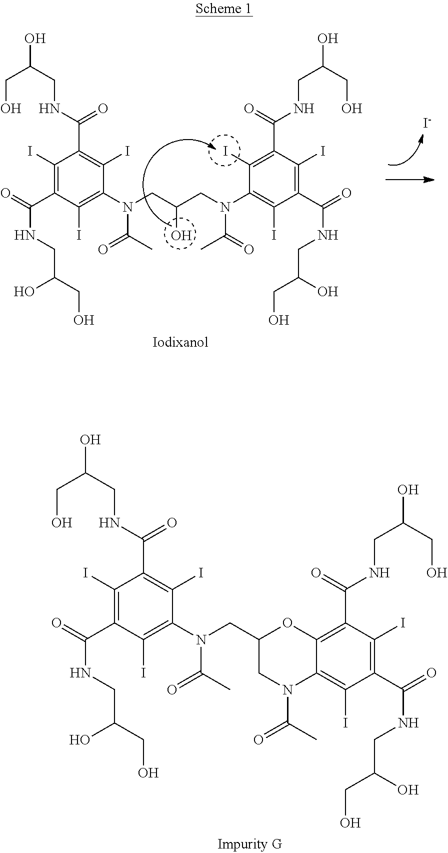

As illustrated, in this cyclization the bond between the denoted iodide atom and the carbon atom in the aromatic ring is replaced with a bond between the oxygen atom of the denoted hydroxyl group and the same carbon atom, resulting in the formation of a 6-member heterocyclic ring fused with one of the aromatic rings (as shown).

In the production of contrast media or agents, purification may be achieved by means of crystallization techniques and/or purification columns, in order to remove impurities from the crude reaction product following completion of the synthetic steps used to prepare it (as described in, for example, U.S. Pat. Nos. 4,396,598 and 5,204,005, which discloses the preparation and/or purification of triiodo-substituted contrast agents, the entire contents of which is encorporated herein by reference for all relevant and consistent purposes). The cost and time involved in such purification operations, including for example the regeneration and/or replacement of purification column packing, is significant. Large amounts of costly resins and large volumes of solutions are also necessary to regenerate the purification column packing between uses. These costs are significant in the production of various contrast media or agents.

Accordingly, there is a need in the art for a method of making triiodinated X-ray contrast agents, such as iodixanol, that provides high conversion or yield of the desired product, while reducing the concentration of impurities that are formed in the reaction mixture. This reduction of impurities in the reaction mixture has the added benefit of reducing the costs associated with subsequent isolation or purification of the desired reaction product.

### BRIEF SUMMARY OF THE DISCLOSURE

Briefly, therefore, the present disclosure is generally directed to an improved process for preparing a triiodinated X-ray contrast agent that limits the formation of fused bicyclic impurities, such as Impurity G, therein. The process comprises contacting in a reaction mixture a triiodo-substituted arylamide having the Structure (I-A):

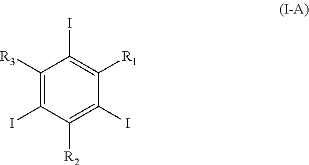

with an alkylating agent in the presence of a base, a suitable solvent, and an alkali metal iodide salt, wherein the molar ratio of the alkali metal iodide salt to the triiodo-substituted arylamide is at least about 1:1. In Structure (I-A), R₁, R₂ and R₃ may be the same or different, and further may be independently selected from —NH—R₅, —C(O)—NH—R₆, or —NH—C(O)—R₆, provided at least one of R₁, R₂ and R₃ has one of the following structures:

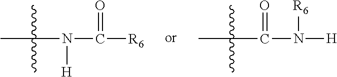

wherein R₅ and R₆ may be the same or different and may be independently selected from hydrogen, or substituted or unsubstituted alkyl, further provided that R₆ is not hydrogen when R₁, R₂ or R₃ has the former structure (i.e., —NH—C(O)—R₆). Preferably, the process is carried out such that the concentration of fused bicyclic impurities in the reaction mixture is less than about 5 area %, relative to the total concentration of the desired reaction product (i.e., the triiodinated X-ray contrast agent) in the reaction mixture (as determined by means known in the art).

More particularly, the present disclosure is directed to an improved process for preparing a triiodinated X-ray contrast agent of Structure (II-B) wherein the formation of fused bicyclic impurities of Structure (II-C) are limited. In the process, as illustrated in Scheme 2, a triiodo-substituted arylamide having Structure (II-A):

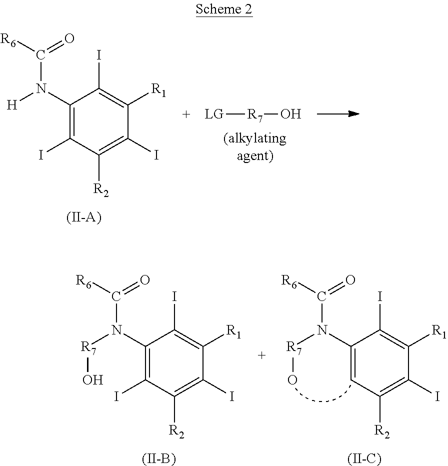

is contacted in a reaction mixture with an alkylating agent, LG-R₇—OH, in the presence of a base, a suitable solvent, and an alkali metal iodide salt, wherein the molar ratio of the alkali metal iodide salt to the triiodo-substituted arylamide is at least about 1:1, and further wherein the concentration of the fused bicyclic impurity (II-C) formed in said reaction mixture is less than about 5 area %, relative to the total concentration of the desired reaction product in the reaction mixture (as determined by means known in the art). In Structures (II-A), (II-B) and (II-C), R₁ and R₂ may be the same or different, and further may be independently selected from —NH—R₅, —C(O)—NH—R₆, or —NH—C(O)—R₆, while R₅ and R₆ may be the same or different and may be independently selected from hydrogen, or substituted or unsubstituted alkyl, provided that R₆ is not hydrogen when R₁ or R₂ has the structure —NH—C(O)—R₆. Additionally, in the alkylating agent, LG-R₇—OH, LG is a leaving group that is displaced during the reaction, while R₇—OH, in the alkylating agent as well as Structures (II-B) and (II-C), is a hydroxyl-substituted methyl, ethyl or propyl substituent, optionally substituted with one or more additional hydrocarbyl or heterohydrocarbyl substituents.

Still more particularly, the present disclosure is directed to such a process wherein a dialkylating agent is used to form an X-ray imaging agent having a dimer structure. In such an embodiment, the reaction may proceed as illustrated in Scheme 3A or Scheme 3B, below:

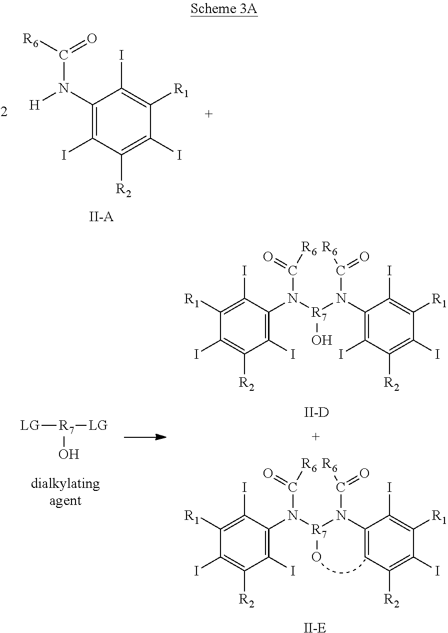

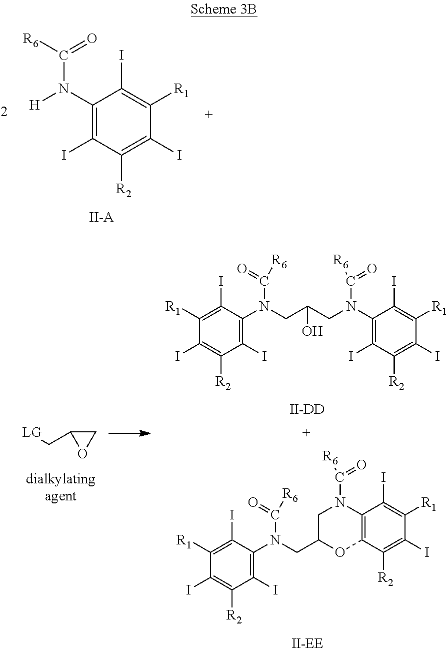

wherein, as illustrated, the dialkylating agent has either (i) two leaving groups that are displaced in the reaction (Scheme 3A), the agent having the formula LG-R₇(OH)-LG, or (ii) only one leaving group that is displaced in the reaction (Scheme 3B), wherein the agent has the formula:

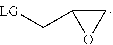

In either of the reaction schemes above, about 2 equivalents of a triiodo-substituted arylamide of Structure (II-A), which may be the same or different (that is, about 2 equivalents of the same arylamide may be used, or about 1 equivalent of 2 different arylamides may be used), are reacted with about 1 equivalent of a dialkylating agent in a reaction mixture that, as detailed above, also comprises a base and an alkali metal iodide salt, the molar ratio of the alkali metal iodide salt to the total triiodo-substituted arylamide being at least about 1:1, to obtain the reaction product of Structure (II-D) or (II-DD), respectively, while limiting the formation of the fused bicyclic impurity (II-E) or (II-EE), respectively, the concentration of (II-E) or (II-EE) being less than about 5 area %, relative to the total concentration of the desired reaction product in the reaction mixture (as determined by means known in the art). Additionally, R₁, R₂, R₅, R₆, R₇ and LG are as set forth above in the preceding paragraph, with the additional provision that the two leaving group (LG) moieties, when present, may be the same or different.

In a particularly preferred embodiment of the present disclosure, the process of the preceding paragraph is carried out to prepare the X-ray contrast agent iodixanol (III-B), while limiting the formation of Impurity G therein (III-C), as illustrated in Scheme 4, below. In the reaction, about two equivalents of the triiodo-substituted arylamide starting compound 5-acetamido-N,N′-bis(2,3-dihydroxylpropyl)-2,4,6-triiodoisophthalamide (III-A), which may alternatively be referred to herein as “Compound A”, is reacted with about one equivalent of a dialkylating agent, and preferably epichlorohydrin, in a reaction mixture comprising a base, a suitable solvent, and an alkali metal iodide salt, the molar ratio of the alkali metal iodide salt to the total triiodo-substituted arylamide being at least about 1:1, to obtain the reaction product of Structure (III-B), while limiting the formation of the fused bicyclic impurity (III-C), the concentration of (III-C) being less than about 5 area %, relative to the total concentration of the desired reaction product in the reaction mixture (as determined by means known in the art).

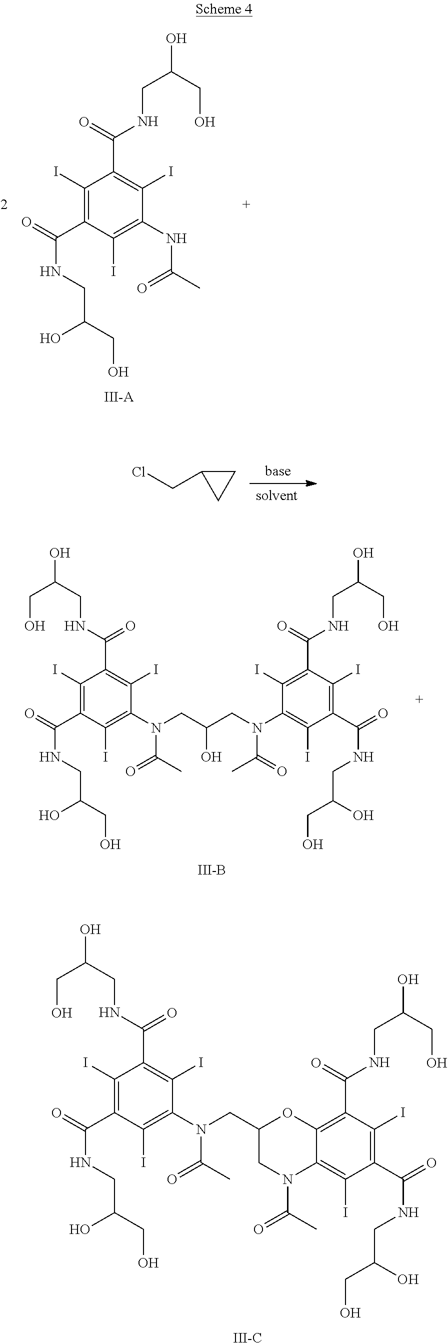

In an alternative embodiment to Scheme 2, the present disclosure is directed to an improved process for preparing a triiodinated X-ray contrast agent of Structure (IV-B) wherein the formation of fused bicyclic impurity of Structure (IV-C) is limited. In the process, as illustrated in Scheme 5, a triiodo-substituted arylamide having Structure (IV-A):

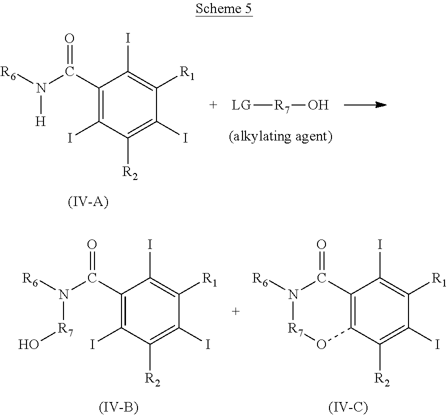

is contacted in a reaction mixture with an alkylating agent, LG-R₇—OH, in the presence of a base, a suitable solvent, and an alkali metal iodide salt, wherein the molar ratio of the alkali metal iodide salt to the triiodo-substituted arylamide is at least about 1:1, and further wherein the concentration of the fused bicyclic impurity (IV-C) is less than about 5 area %, relative to the total concentration of the desired reaction product in the reaction mixture (as determined by means known in the art). In Structures (IV-A), (IV-B) and (IV-C), R₁ and R₂ may be the same or different, and further may be independently selected from —NH—R₅, —C(O)—NH—R₆, or —NH—C(O)—R₆, while R₅ and R₆ may be the same or different and may be independently selected from hydrogen, or substituted or unsubstituted alkyl, provided that R₆ is not hydrogen when R₁ or R₂ has the structure —NH—C(O)—R₆. Additionally, in the alkylating agent, LG-R₇—OH, LG is a leaving group that is displaced during the reaction, while R₇—OH, in the alkylating agent as well as Structures (IV-B) and (IV-C), is a hydroxyl-substituted methyl, ethyl or propyl substituent, optionally substituted with one or more additional hydrocarbyl or heterohydrocarbyl substituents.

In a preferred embodiment of one or more of the above noted processes, the reaction mixture comprises a mixed solvent system comprising a non-aqueous solvent and water, wherein the volume ratio thereof is greater than 1:1, and preferably is about 2:1, and less than about 10:1. In a more preferred embodiment, the mixed solvent system comprises dimethylacetamide (DMAc) and water, and in a still more preferred embodiment the mixed solvent system comprises these components in a volume ratio of about 2:1, respectively.

### DETAILED DESCRIPTION OF THE DISCLOSURE

As further detailed herein below, it has been discovered that the addition of an alkali metal iodide salt to the reaction mixture used to alkylate triiodo-substituted arylamide compounds, such as for example phenylamide compounds, in order to form compounds suitable for use as X-ray contrast agents, acts to limit or reduce the formation of fused bicyclic, impurities therein. In particular, it has been discovered that the addition of an alkali metal iodide salt, such as potassium iodide, to the reaction mixture used to form iodixanol acts to limit or reduce the formation of Impurity G therein.

Without being held to any particular theory, and as further illustrated in Scheme 1 below, it is generally believed that a side-reaction that occurs in the reaction mixture, after the desired alkylated reaction product (iodixanol in the illustration) is formed therein, results in the formation of a fused bicyclic impurity (Impurity G in the illustration) and produces an iodide ion as a by-product. It has been discovered that the addition of an alkali metal iodide salt, and more particularly at least a 1:1 equivalent of the alkali metal iodide salt relative to the triiodo-substituted arylamide starting compound, act to inhibit this unwanted side-reaction. As a result, the amount of impurities, and specifically fused bicyclic impurities (such as Impurity G), may be limited, thus simplifying the subsequent isolation or purification that is to be performed before obtaining the desired reaction product.

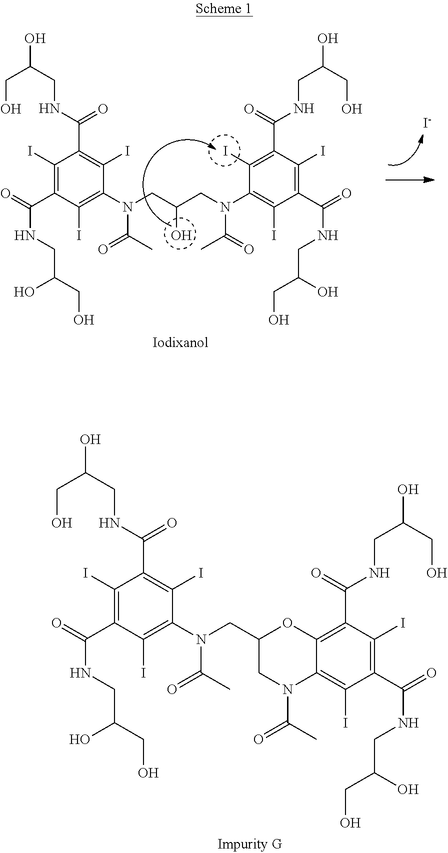

### I. X-RAY CONTRAST AGENTS

As previously noted, the present disclosure is generally directed to an improved process for preparing triiodinated X-ray contrast agents that limits the formation of fused, bicyclic impurities therein. The process comprises alkylating a triiodo-substituted arylamide having Structure (I-A), below, with an alkylating agent in the presence of a base, a suitable solvent, and an alkali metal iodide salt, wherein the molar ratio of the alkali metal iodide salt to the triiodo-substituted arylamide is at least about 1:1, and in various embodiments may be about 1.25:1, about 1.5:1, about 1.75:1, about 2:1, about 2.25:1 or even about 2.5:1, the molar ratio for example being within the range of about 1:1 to about 2.5:1, or about 1:1 to about 2:1. Suitable alkali metal iodide salts may be selected, for example, from essentially any alkali metal iodide salt commercially available or that may be readily prepared, including for example sodium iodide, potassium iodide, lithium iodide and cesium iodide, with potassium iodide being preferred for one or more embodiments.

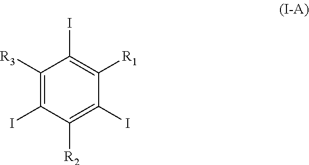

In Structure (I-A), R₁, R₂ and R₃ may be the same or different, and further may be independently selected from —NH—R₅, —C(O)—NH—R₆, or —NH—C(O)—R₆, provided at least one of R₁, R₂ and R₃ has one of the following structures:

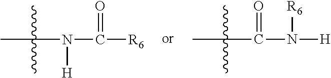

wherein R₅ and R₆ may be the same or different and may be independently selected from hydrogen, or substituted or unsubstituted alkyl, and in various embodiments may be substituted or unsubstituted lower alkyl (e.g., methyl, ethyl, propyl, butyl, pentyl, etc., optionally substituted with, for example, one or more heteroatom-containing groups, such as hydroxyl, alkoxy, amino or amido), and further provided that R₆ is not hydrogen when R₁, R₂ or R₃ has the former structure (i.e., —NH—C(O)—R₆). In one preferred embodiment, at least one of R₁, R₂ and R₃ in the triiodo-substituted arylamide of Formula (I) has the structure:

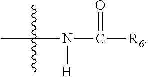

In a more preferred embodiment, however, only one of R₁, R₂ and R₃ has the above-noted structure, the other two having the structure:

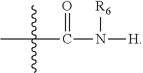

Process conditions, and in particular the concentration or amount of the alkali metal iodide salt used therein (e.g., molar ratio of the metal salt to the triiodo-substituted arylamide starting compound), may be optimized in order to limit, and preferably substantially prevent, the formation of fused bicyclic impurities, such as Impurity G, in the reaction mixture. Desirably, the concentration of such impurities in the reaction mixture is less than about 5 area %, relative to the total concentration of the desired reaction product in the reaction mixture (as determined by means known in the art, including for example high performance liquid chromatography (HPLC) techniques), and preferably is less than about 4 area %, about 3 area %, about 2 area %, or even about 1 area %, the concentration for example being within the range of about 5 area % and about 1 area %, or about 3 area % and about 2 area %.

As generally illustrated in Scheme 2 below, for the representative compound of Structure (II-A), in the present reaction a N atom that is part of the amide functionality on the ring is alkylated to replace the H atom bound thereto with a the substituent R₇—OH, derived from the alkylating agent, to obtain the compound of Structure (II-B).

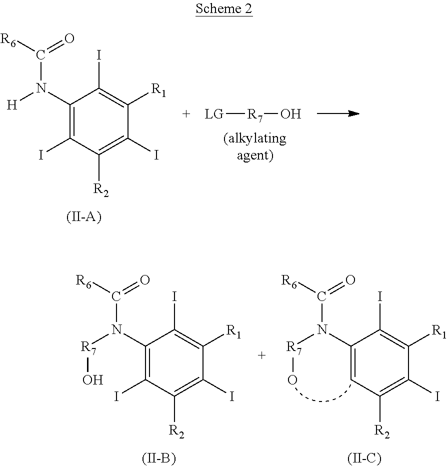

In Structures (II-A), (II-B) AND (II-C), R₁, R₂ and R₆ are as previously defined above, while R₇—OH is, in general, a hydroxyl-substituted methyl, ethyl or propyl substituent, optionally substituted with one or more additional hydrocarbyl or heterohydrocarbyl substituents.

In an alternative embodiment to Scheme 2, and as illustrated by Structure (IV-A) in Scheme 5 below, the N atom that is part of the amide functionality may be bound to the aromatic ring through a carbonyl carbon, rather than being directly bound thereto. In the process, however, this N atom may still be alkylated, as illustrated by Structure (IV-B), to replace the H atom bound thereto with the substituent, R₇—OH, which as detailed above is derived from the alkylating agent, under the noted process conditions, in order to limit the possible formation of fused bicyclic impurities, as illustrated by Structure (IV-C). In this alternative embodiment, and as illustrated in Scheme 5, a triiodo-substituted arylamide of Structure (IV-A):

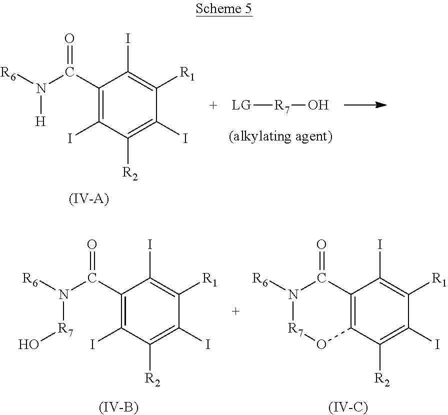

In Structures (IV-A), (IV-B) AND (IV-C), R₁, R₂ and R₆ are as previously defined above, while R₇—OH is, in general, a hydroxyl-substituted methyl or ethyl substituent, optionally substituted with one or more additional hydrocarbyl or heterohydrocarbyl substituents.

In this regard it is to be noted that Schemes 2 and 5, and the compounds therein, are provided for illustration purposes only, and therefore should not be viewed in a limiting sense. For example, in alternative embodiments, two or three amide groups may be present on the ring of Structure (II-A) and/or (IV-A), and, if reacted with multiple molar equivalents (e.g., two or three) of an alkylating agent, two or three of these amide groups be alkylated in the compound of Structure (II-B) and/or (IV-B).

In this regard it is to be further noted that, in yet other alternative embodiments as illustrated in Scheme 3A or Scheme 3B, below, a dialkylating agent, may be used with multiple molar equivalents (e.g., about two) of one or more starting triiodo-substituted arylamide compounds (e.g., about two moles of a single starting compound per mole of dialkylating agent, or about one more of two different starting compounds per mole of dialkylating agent), consistent with the details set forth above, to obtain a dimer or dimerized reaction product of Structure (II-D or II-DD), such as iodixanol, while limiting the formation of the fused bicyclic impurity (II-E or II-EE), as detailed above.

As illustrated in Scheme 3A or Scheme 3B, below, the reaction may proceed using a dialkylating agent that has either (i) two leaving groups that are displaced in the reaction (Scheme 3A), the agent having the formula LG-R₇(OH)-LG, or (ii) only one leaving group that is displaced in the reaction (Scheme 3B), wherein the agent has the formula, for example:

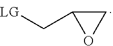

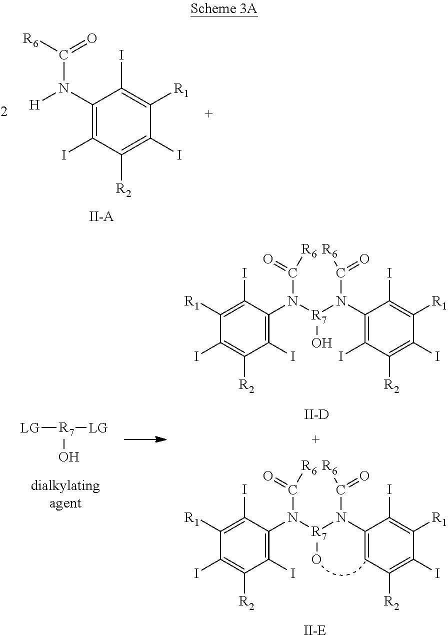

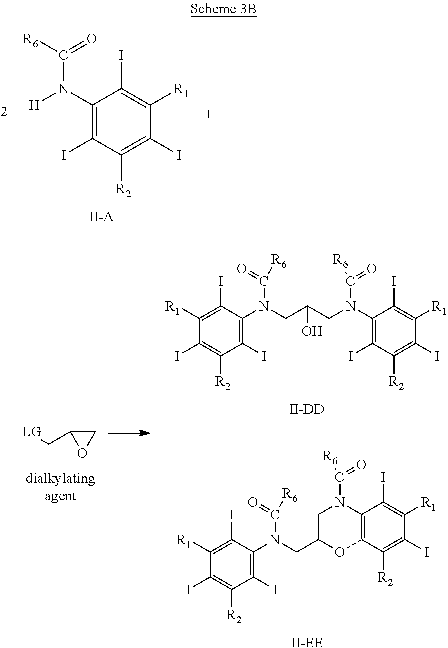

In this regard, it is to be noted that, in Structures (II-A), (II-D), (II-DD), (II-E) and (II-EE), substituents R₁, R₂, R₅, R₆, R₇, and LG are as set forth above in the preceding paragraph, with the additional provision that the two leaving group (LG) moieties may be the same or different. It is to be additionally noted that the heterocyclic alkylating agent may be other than illustrated above without departing from the scope of the present disclosure. For example, in one or more alternative embodiments, the number of atoms (e.g., carbon atoms) in the chain attaching the leaving group (LG) to the heterocylic ring, and/or the size of the heterocyclic ring itself, may be more or less than illustrated above, provided the leaving group (LG) is separated by 5, 6 or 7 atoms from the carbon-iodide (C-1) bond, such that a 5, 6 or 7-member ring may form upon loss of the leaving group and the iodide atom.

In one particularly preferred embodiment of Scheme 3B, and as further illustrated in Scheme 4 below, the X-ray contrast agent iodixanol (11′-B) may be prepared, while limiting the formation of Impurity G (111-C) therein. In the reaction, about two equivalents of the triiodo-substituted arylamide starting compound 5-acetamido-N,N′-bis(2,3-dihydroxylpropyl)-2,4,6-triiodoisophthalamide (III-A), which may alternatively be referred to herein as “Compound A”, is reacted with a dialkylating agent (as further detailed herein below), and preferably epichlorohydrin, in the presence of a base, such as sodium hydroxide, and a suitable solvent, wherein the molar ratio of the alkali metal iodide salt to the total triiodo-substituted arylamide is at least about 1:1, and further wherein the concentration of the fused bicyclic impurity (II-E) in the reaction mixture is less than about 5 area %, relative to the total concentration of the desired reaction product in the reaction mixture (as determined by means known in the art, including for example high performance liquid chromatography (HPLC) techniques), and preferably is less than about 4 area %, about 3 area %, about 2 area %, or even about 1 area %, the concentration for example being within the range of about 5 area % and about 1 area %, or about 3 area % and about 2 area %.

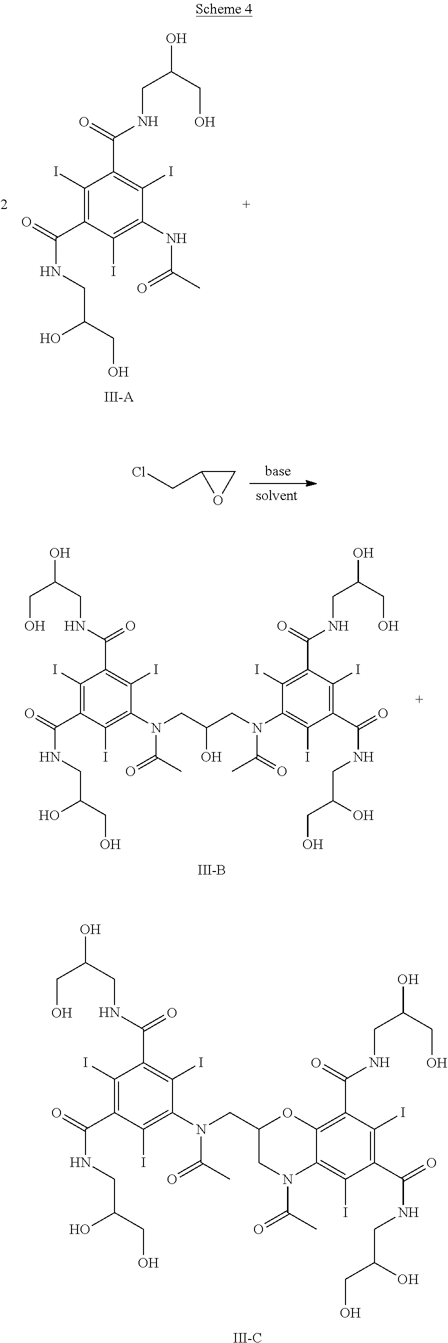

The compounds generally encompassed by starting Structures (I-A), (II-A), (IV-A), and (III-A) may be obtained commercially, or alternatively they may be prepared using processes and methodologies generally known in the field. For example, (III-A), which may alternatively be referred to herein as Compound A, may be prepared using techniques generally known in the art, such as for example by the process disclosed in U.S. Pat. No. 5,705,692 (the entire contents of which are incorporated herein by reference for all relevant and consistent purposes), and more specifically the process disclosed in Example 1 therein.

It is to be noted that the present process may in general be utilized to more effectively or efficiently prepare essentially any triiodinated X-ray contrast agent in which the formation of fused bicyclic impurities may occur. Such triiodinated X-ray contrast agents may be generally identified by the presence of a substituted hydroxyl substituent on the ring thereof, which is capable of forming a 5-member, 6-member or 7-member ring; stated another way, these agents may be generally identified as those having for example the structure (IV-B) or (IV-C), wherein R₇ is a hydroxyl-substituted methyl (6-member ring) or ethyl (7-member ring), or having the structure (II-B) or (II-C), wherein R₇ is a hydroxyl-substituted methyl (5-member ring), ethyl (6-member ring), or propyl (7-member ring).

### II. SOLVENT

As noted above, in accordance with the present disclosure, the alkylation of a triiodo-substituted arylamide is carried out in the presence of a suitable solvent. Selection of a suitable solvent may be made based on such factors as the solubility of the starting compounds or other reagents and/or the solubility of the resulting reaction products or byproducts (i.e., impurities) therein. For example, solubility of the desired reaction product and undesirable reaction byproducts is a consideration, because differences in solubility in the solvent may aid with subsequent isolation and/or purification of the desired reaction product.

In general, however, suitable solvents include for example water, as well as polar organic, or polar aprotic, solvents. Suitable solvents include, for example, methanol, 2-methoxyethanol, isopropanol, dimethylacetamide (DMAc), dimethyl sulfoxide (DMSO), dimethyl formamide (DMF), tetrahydrofuran (THF), and acetonitrile (ACN), as well as a mixture of two or more thereof. In one preferred embodiment, however, the solvent is a mixed solvent system that comprises a non-aqueous solvent and water, and more preferably a mixed solvent system wherein the non-aqueous solvent is the major component and water is the minor component, provided sufficient water is present to ensure dissolution or solubility of one or more of the reaction mixture components and/or the reaction product (e.g., iodixanol) that is formed.

In this regard it is to be noted that, as used herein, “mixed solvent system” refers to a solvent system comprising a non-aqueous solvent and water, wherein the concentration of water therein is more than just a trace amount (or is above the level commonly associated with being an impurity). In such an embodiment, the non-aqueous solvent may in general be selected from among those that are miscible with water, and more particularly are polar aprotic solvents (e.g., dimethylacetamide (DMAc), dimethyl sulfoxide (DMSO), dimethyl formamide (DMF), tetrahydrofuran (THF), and acetonitrile (ACN), as well as a mixture thereof). In one or more preferred embodiments, the components of the solvent system will be selected in order to obtain a homogeneous or single-phase reaction solution or reaction mixture upon completion of the reaction (as determined, for example, upon expiration of a designated reaction time limit or upon reaching some minimum reaction product concentration in the reaction solution or mixture, as further detailed elsewhere herein). It is generally believed that a single-phase reaction solution enables the reaction product to be more easily isolated, and/or for the undesirable reaction byproducts to be more easily removed. For example, experience to-date has shown that, in the preparation of iodixanol, the combination of DMAc and water advantageously results in a single phase or homogeneous reaction mixture (after the reaction is determined to be completed). While the presence of a two-phase reaction mixture is not necessarily problematic, it may create the need for additional steps during isolation and/or purification of the reaction product.

In the embodiments wherein a mixed solvent system is employed, the mixed solvent system may be one having a volume ratio of a non-aqueous solvent to water that is greater than 1:1 and less than about 10:1, or greater than 1:1 and less than about 5:1, and in various embodiments may be greater than about 1.25:1, about 1.5:1, about 1.75:1, about 2:1, about 2.25:1, about 2.5:1, or even about 3:1, and less than about 10:1 or about 5:1. In one or more preferred embodiments, however, the volume ratio is between 1:1 and about 5:1, or between 1:1 and about 3:1, or between 1.5:1 and about 2.5:1, or between about 1.75:1 and about 2.25:1, with the ratio of about 2:1 being most preferred in one or more embodiments. In one particularly preferred embodiment, the solvent system is a mixture of DMAc and water, and more preferably comprises, or consists essentially of, DMAc and water, wherein the volume ratio of these two components is between about 1.75:1 and about 2:25:1, and most preferably is about 2:1.

### III. ALKYLATING AGENTS AND BASES

As previously noted, the triiodo-substituted X-ray contrast agents of the present disclosure are produced by an alkylation reaction, wherein a triiodo-substituted arylamide is contacted with an alkylation agent in the presence of a solvent, a base and an alkali metal iodide salt. A number of alkylating agents are generally known in the art, and selection from among these for use in the process of the present disclosure may be made based on such consideration as, for example: (i) sufficient reactivity with the amide functionality, and more particularly the nitrogen atom of the amide functionality, of the triiodo-substituted arylamide compound, such that alkylation may occur; (ii) appropriate composition of the alkyl group, which is transferred from the alkylating agent to the triiodo-substituted arylamide compound; and/or (iii) sufficient solubility in the solvent.

As previously noted, in accordance with the present process, the triiodo-substituted arylamide starting compound is reacted with an alkylating agent, such as LG-R₇—OH, or dialkylating agent, such as LG-R₇(OH)-LG or an agent having the structure:

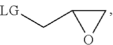

wherein R₇ is methyl, ethyl or propyl, optionally substituted with one or more additional hydrocarbyl or heterohydrocarbyl substituents. In the alkylating agent, LG is essentially any leaving group that is displaced from the remaining portion of the alkylating agent during the reaction. This leaving group (or groups, in some embodiments of the dialkylating agent, wherein two groups are displaced), may more specifically be a heteroatom, or a heteroatom-containing moiety, such as for example a halogen atom (e.g., fluoro, chloro, bromo, etc.), or a hydroxyl group, or an alkoxy group (e.g., C₁₋₁₀ or C₁₋₅, including for example, methoxy, ethoxy, propoxy, butoxy, etc.), or a combination thereof.

Also as previously noted, in various embodiments the agent may be either a mono-alkylating agent, or a dialkylating agent (the agent for example having two reactive sites and thus enabling two molecules of a triiodo-substituted arylamide to be linked together). In a particularly preferred embodiment, the alkylating agent may selected from the group consisting of monohalo- or dihalo-substituted alkanols or dialkanols (e.g., 1,3-dihalo-2-propanol, such as 1,3-dichloro-2-propanol, or 1-halo-2,3-propane diol, such as 1-chloro-2,3-propane diol), any of which may optionally be further substituted with an alkoxy group, such as a methoxy group (e.g., 1-halo-3-alkoxy-propanol, such as 1-chloro-3-methoxy-2-propanol), as well as various halo-substituted heterocycloalkyl compounds (e.g., epichlorohydrin or glycidol).

In addition to the alkylating (or dialkylating) agent and the starting triiodo-substituted arylamide, as well as the solvent, the reaction mixture additionally comprises a base. Generally speaking, essentially any base may be used that will enable the alkylating reaction to be carried out in a satisfactory way (e.g., sufficient reaction product yield, and/or purity). Typically, however, the base will be selected from known alkali or alkali earth metal hydroxides (e.g., sodium hydroxide, potassium hydroxide, lithium hydroxide, cesium hydroxide, etc.), alkali or alkali earth metal carbonates (e.g., sodium carbonate, potassium carbonate, lithium carbonate, cesium carbonate, etc.), and strong organic bases (i.e., bases which act to raise the pH of the reaction mixture to about 10 or more, as detailed elsewhere herein below).

As previously noted, the molar ratio of the starting compound (I.e., the compound of Structure (I-A), (II-A), (IV-A), and/or (III-A)), the alkylating or dialkylating agent, and/or base, may be determined or optimized using means generally known in the art, in order to maximize purity and/or yield of the desired product. Typically, however, the molar ratio of starting compound to alkylating agent will be between about 1:3 (e.g., when multiple sites on the triiodo-substituted compound are to be alkylated) and about 2:1 (e.g., when two molecules of the starting compound are reacted with a single molecule of a dialkylating agent, in order to form a dimer), with ranges of about 1:2 to about 2:1, or about 1:1 to about 2:1, or about 1.5:1 to about 2:1, being more commonly employed. In one particular embodiment, wherein about 2 molar equivalents of the starting compound are to be reacted with or linked by means of about 1 molar equivalent of a dialkylating agent, a slight molar excess of the dialkylating agent may be used, to for example offset the slight consumption of dialkylating agent by the base. Accordingly, in such an embodiment the molar ratio of the starting compound to the dialkylating agent may be about 2:1.1, about 2:1.15, or about 2:1.2.

### IV. REACTION CONDITIONS AND PROCESS STEPS

The process of the present disclosure generally involves forming a reaction mixture comprising the solvent, the base, the alkylating (or dialkylating) agent, the triiodo-substituted arylamide starting compound, and the alkali metal iodide salt. In the process, the order of addition of the components is not narrowly critical; that is, the base, the alkylating agent and triiodo-substituted compound and alkali metal iodide salt may be added to the solvent in essentially any order. Preferably, however, the reaction mixture is formed by initially mixing or slurrying together the triiodo-substituted arylamide compound, the base and the solvent. After agitating this mixture or slurry for a given period of time (e.g., at least about 30 minutes, 60 minutes or even 90 minutes), the alkylating agent and alkali metal iodide salt are added. The pH of the reaction mixture may optionally be adjusted before or after addition of the alkylating agent and/or alkali metal iodide salt as needed, in order to maximize or optimize the reaction (e.g., to increase reaction product yield and/or limit the formation of impurities). In one or more embodiments, the pH of the reaction mixture may be monitored and adjusted before or during the reaction, to ensure the pH is within the range of about 10 and about 14, or about 11 and about 13 (as determined using means known in the art).

Once the reaction mixture is formed, the reaction mixture may be heated or cooled as needed to maintain the reaction mixture within a desired temperature range for a desired period of time. For example, in one embodiment, the temperature of the reaction mixture will be maintained within the range of from about 0° C. to about 75° C., or from about 5° C. to about 60° C., or from about 10° C. to about 50° C., or from about 20° C. to about 40° C.

The reaction time, or more specifically the time the reaction mixture is maintained within the desired temperature range, may be set based on a number of factors, such as the concentration of the desired reaction product in the reaction mixture or the concentration of unwanted impurities or byproducts in the reaction mixture (as determined using means generally known in the art, including for example withdrawing an aliquot of the reaction mixture and subjecting it to a known analytical method, such as HPLC, to measure the concentration of the desired reaction product or unwanted impurity or byproduct therein). Typically, however, the reaction time will be between about 5 hours and about 75 hours, or between about 10 hours and about 50 hours, or between about 15 hours and about 25 hours.

In this regard it is to be noted that the order of addition, the reaction temperature, reaction mixture pH, and/or the reaction time or duration, may be other than herein described without departing from the scope of the present disclosure.

### V. REACTION PRODUCT ISOLATION AND YIELD

Once the reaction has reached the desired end point (as determined, for example, by passage of a sufficient amount of time or by means of analytical analysis), the reaction may be stopped or quenched using means generally known in the art. For example, in one particular embodiment, the reaction may be stopped or quenched by the addition of an appropriate amount of an acid (e.g., a hydrochloric acid). Additionally, means generally known in the art may be used to take the reaction mixture forward, in order to isolate and purify the desired reaction product as needed. For example, in one particular embodiment, the reaction mixture is processed using means generally known in the art (e.g., distillation, solvent separation or extraction, etc.) to remove any non-aqueous component of the solvent (e.g., DMAc) that may be present. The remaining, essentially aqueous, solution may then be further processed by adding additional water (in order, for example, to ensure all components therein are thoroughly dissolved), followed by subjecting the solution to de-salting and deionizing techniques generally known in the art, prior to final purification of the reaction product.

By the proper selection of reaction mixture components, such as solvent (or solvent system components, and the relative molar ratios therebetween) or molar ratio of alkali metal iodide salt to triiodo-substituted arylamide starting compound, the process of the present disclosure enables the desired reaction product to be obtained in a yield of about 50%, about 55%, about 60%, about 65%, about 70%, or more, based on the total weight of the reaction product mixture (i.e., the mixture obtained upon completion of the reaction to form the reaction product), the yield for example being in the range of about 50% to about 70%, or about 55% to about 65%. The process of the present disclosure additionally enable the desired reaction product (e.g., iodixanol) to be obtained, after the reaction product has been isolated and purified by means generally known in the art, having an overall impurity concentration (including Impurity G, or other fused bicyclic impurities) of less than about 5 area %, about 4 area %, about 3 area %, about 2 area %, or even less than about 1 area % (relative to the reaction product itself), as determined by means generally known in the art. Stated another way, the reaction product (e.g., iodixanol), after isolation and purification by means generally known in the art, may be obtained having a purity of at least about 95 area %, about 96 area %, about 97 area %, about 98 area %, about 99 area %, or more.

Additionally, or alternatively, the process of the present disclosure advantageously (i) enables the concentration of one or more undesirable reaction impurities or byproducts (e.g., difficult to remove impurities, such as one or more starting compounds or over-alkylated reaction byproducts, as well as, in the case of iodixanol, Impurity G and/or iohexyl) in the reaction product mixture to be reduced by limiting their formation, and/or (ii) simplifies subsequent purification of the reaction product (by, for example, eliminating or reduced the concentration of hard to remove impurities in the reaction product mixture, such as those previously noted). For example, by proper selection of the mixed solvent system, removal of impurities, such as unreacted starting components (such as the triiodo-substituted arylamide, or Compound A in the case of iodixanol), and/or reaction byproducts or salts (e.g., iohexyl, when the desired reaction product is iodixanol), and/or hard to remove impurities (e.g., over-alkylated compounds, and/or Impurity G), may be simplified, by for example preventing or limiting their formation, and/or ensuring that such impurities (such as, in the case of iodixanol, starting Compound A, or over-alkylated compounds, or iohexyl) remain in solution, with or without the reaction product (i.e., the reaction product may or may not remain in solution).

In this regard it is to be noted that the reaction yield, and/or purity (or impurity concentration), as well as the concentration and type of impurities present in the reaction product mixture, may be other than herein described without departing from the scope of the intended invention.

### VI. DEFINITIONS

The compounds described herein may have asymmetric centers. Compounds of the present disclosure containing an asymmetrically substituted atom may be isolated in optically active or racemic form. All chiral, diastereomeric, racemic forms and all geometric isomeric forms of a structure are intended, unless the specific stereochemistry or isomeric form is specifically indicated. All processes used to prepare compounds of the present disclosure and intermediates made therein are considered to be part of the present disclosure.

As used herein, “optional” or “optionally” means that the subsequently described event or circumstance may or may not occur, and that the description includes instances where the event or circumstance occurs and instances where it does not.

The term “amido” as used herein includes substituted amido moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, phosphorous-oxo acid, sulfur-oxy acid, hydroxyl, oxy, mercapto, and thio substituents.

The term “amino” as used herein includes substituted amino moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, phosphorous-oxo acid, sulfur-oxy acid, hydroxyl, oxy, mercapto, and thio substituents.

The terms “aryl” or “ar” as used herein, alone or as part of another group, denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “arylamide” as used herein refers to aromatic compounds having one or more amide or amido substituents thereon. Phenyl and substituted phenyl are the more preferred amide or amido substituted rings.

The terms “halogen” or “halo” as used herein alone or as part of another group refer to chlorine, bromine, fluorine, and iodine.

Unless otherwise indicated, the “alkyl” groups described herein are preferably lower alkyl containing from one to 10 carbon atoms in the principal chain, and up to 20 carbon atoms. They may be straight or branched chain or cyclic (e.g., cycloalkyl) and include methyl, ethyl, propyl, isopropyl, butyl, pentyl, hexyl and the like. Accordingly, the phrase “C₁₋₂₀ alkyl” generally refers to alkyl groups having between about 1 and about 20 carbon atoms, and includes such ranges as about 1 to about 15 carbon atoms, about 1 to about 10 carbon atoms, or about 1 to about 5 carbon atoms.

The term “substituted” as in “substituted arylamide” or “substituted alkyl” and the like, means that in the group in question (i.e., the amine, the alkyl, or other moiety that follows the term), at least one hydrogen atom bound to a nitrogen atom or carbon atom, respectively, is replaced with one or more substituent groups such as hydroxy, alkoxy, alkylthio, phosphino, amino, halo, silyl, and the like. When the term “substituted” introduces a list of possible substituted groups, it is intended that the term apply to every member of that group. That is, the phrase “substituted alkyl, alkenyl and alkynyl” is to be interpreted as “substituted alkyl, substituted alkenyl and substituted alkynyl.” Similarly, “optionally substituted alkyl, alkenyl and alkynyl” is to be interpreted as “optionally substituted alkyl, optionally substituted alkenyl and optionally substituted alkynyl.”

The term “alkanol” refers to an alkyl group having a hydroxy group or substituent thereon. The term “dialkanol” refers to an alkyl group having two hydroxy groups or substituents therein.

The modifiers “hetero” and “heteroatom-containing”, as in “heteroalkyl” or “heteroatom-containing group” refer to a molecule or molecular fragment in which one or more carbon atoms is replaced with a heteroatom. Thus, for example, the term “heteroalkyl” refers to an alkyl group that contains a heteroatom, while “heterocycloalkyl” reference to a cycloalkyl group that contains a heteroatom. When the term “heteroatom-containing” introduces a list of possible heteroatom-containing groups, it is intended that the term apply to every member of that group.

As illustrated below, the term “fused bicyclic” generally refers to a compound that includes two rings therein, and further wherein each of the rings in the compound share two ring atoms (e.g., carbon atoms or heteroatoms, as highlighted by the dashed-circles below), one or more of the atoms in the ring being a heteroatom. Optionally, when a heteroatom is present, the term “fused hetero-bicyclic” may be used.

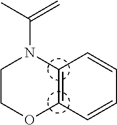

### EXAMPLES

The following non-limiting examples are provided to further illustrate the present disclosure.

### Example 1

### Preparation of Iodixanol without KI (Control)

15 grams (0.02 moles) of 5-acetamido-N,N′-bis(2,3-dihydroxypropyl)-2,4,6-triiodoisophthalamide (Compound A) was charged to a 100 ml round bottom flask fitted with a mechanical stirrer, along with 30 ml of DMAc (Fisher) and 15 ml of water. While stirring at room temperature, 1.44 ml (0.014 moles) of 10 N NaOH (Fisher) solution was added. Stirring was continued for 30 minutes at room temperature, to allow the mild exotherm to subside. Epichlorohydrin (0.53 ml, 0.007 moles, Acros) was then added. Stirring was continued, and aliquots were taken after 16 hours, 19 hours and 45 hours and subjected to HPLC analysis. The results of the HPLC analysis are presented in Table 1, below (the balance of each analyzed sample being over-alkylated impurities).

TABLE 1

| | Concentration | | |
|---|---|---|---|
| Component | 16 hours | 19 hours | 45 hours |
| Compound A | 43.60% | 39.05% | 38.44% |
| Iohexol | 2.84% | 3.49% | 2.96% |
| Impurity G | 0.84% | 1.40% | 3.86% |
| Iodixanol | 47.54% | 52.0% | 50.65% |
| Total | 94.82% | 95.94% | 95.91% |

### Example 2

### Preparation of Iodixanol with KI Added

Example 1 was repeated, this time differing only by the addition of 6.67 g (0.04 moles, Aldrich) of potassium iodide (KI) to the reaction mixture immediately after addition of the NaOH. As in Example 1, aliquots were taken after 16 hours, 19 hours and 45 hours and subjected to HPLC analysis. The results are presented in Table 2, below.

TABLE 2

| | Concentration | | |
|---|---|---|---|
| Component | 16 hours | 19 hours | 45 hours |
| Compound A | 46.56% | 41.50% | 40.69% |
| Iohexol | 5.58% | 6.27% | 5.6% |
| Impurity G | 0.29% | 0.41% | 0.99% |
| Iodixanol | 42.54% | 47.93% | 49.29% |
| Total | 94.97% | 96.11% | 96.57% |

### Example 3

### Preparation of Iodixanol with KI Added

20 grams (0.026 moles) of 5-acetamido-N,N′-bis(2,3-dihydroxypropyl)-2,4,6-triiodoisophthalamide (Compound A) was charged to a 100 ml round bottom flask fitted with a mechanical stirrer, along with 40 ml of DMAc (Fisher) and 20 ml of water. While stirring at room temperature, 1.874 ml (0.019 moles) of 10 N NaOH (Fisher) solution was added, followed by 4.32 g (0.026 moles) of KI. Stirring was continued for 30 minutes at room temperature, to allow the mild exotherm to subside. Epichlorohydrin (0.69 ml, 0.009 moles, Acros) was then added. Stirring was continued, and aliquots were taken after 19 hours, and 90 hours and subjected to HPLC analysis to measure Impurity G content only. The results are presented in Table 3, below.

TABLE 3

Concentration

| Component | 19 hours | 90 hours |
|---|---|---|
| Impurity G | 0.47% | 2.58% |

### Example 4

### Preparation of Iodixanol with KI Added

20 grams (0.026 moles) of 5-acetamido-N,N′-bis(2,3-dihydroxypropyl)-2,4,6-triiodoisophthalamide (Compound A) was charged to a 100 ml round bottom flask fitted with a mechanical stirrer, along with 40 ml of DMAc (Fisher) and 20 ml of water. While stirring at room temperature, 1.69 ml (0.017 moles) of 10 N NaOH (Fisher) solution was added, followed by 4.32 g (0.026 moles) of KI. Stirring was continued while the mild exotherm subsided and the temperature returned to room temperature. Epichlorohydrin (0.63 ml, 0.008 moles, Acros) was then added. Stirring was continued, and aliquots were taken after 22.5 hours, 46.5 hours, and 72 hours and subjected to HPLC analysis to measure Impurity G content only. The results are presented in Table 4, below.

TABLE 4

| | Concentration | | |
|---|---|---|---|
| Component | 22.5 hours | 46.5 hours | 72 hours |
| Impurity G | 0.28% | 0.62% | 0.88% |

### Example 5

### Compound A Coupling—Low Conversion (DMAc/H₂O) with KI or NaI

Reactions were carried out to see if KI, NaI or KCl could suppress the formation of impurity G in low conversion DMAc/H₂O coupling reactions. The results, presented in Table 5 below, indicate that both KI and NaI are effective at suppressing the formation of this impurity, with NaI appearing to have the greatest effect.

TABLE 5

| | | | | | | | Over- | |
|---|---|---|---|---|---|---|---|---|
| | Base | | Solvent | Cmpd | | | alkylated | |
| Exp. | (eq.) | Epi. (eq.) | (mL/g) | A | Iohexol | Iodixanol | Imp. | Imp. G |
| 3954 × 98 | 0.72 | 0.34 | 3 | | | | | |
| 19 hr. | | Kl = 1 | | 41.35 | 5.83 | 49.21 | 1.87 | 0.47 |
| | | eq. | | | | | | |
| 90 hr. | | | | 38.03 | 4.32 | 51.89 | 2.21 | 2.58 |
| 3954 × 104 | 0.72 | 0.34 | 3 | | | | | |
| 20 hr. | | Kl = 1 | | 48.54 | 5.11 | 42.76 | 1.38 | 0.22 |
| | | eq | | | | | | |
| 43 hr. | | | | 44.68 | 4.84 | 47.08 | 1.61 | 0.66 |
| 3954 × 105 | 0.72 | 0.34 | 3 | | | | | |
| 20 hr. | | Nal = 1 | | 48.63 | 5.34 | 42.72 | 1.38 | 0.25 |
| | | eq | | | | | | |
| 43 hr. | | | | 45.59 | 4.83 | 46.58 | 1.34 | 0.56 |
| 3954 × 108 | 0.80 | 0.38 | 3 | | | | | |
| 20.5 hr. | | | | 34.60 | 4.08 | 54.42 | 3.33 | 2.42 |
| 42.5 hr. | | Kl = 1.0 | | 32.53 | 3.25 | 53.66 | 3.81 | 5.84 |
| | | eq | | | | | | |
| 49.5 hr. | | | | 32.50 | 3.26 | 53.14 | 3.56 | 6.32 |
| 119 hr. | | | | 32.80 | 3.21 | 51.61 | 3.44 | 6.36 |

Note
that Kl was added in this last experiment at 42.5 hours to see if formation of impurity G could be reversed.

### Example 6

### Compound A Coupling—High Conversion (DMAc/H₂O) with KI or NaI

Reactions were run to see if adding KI or NaI could suppress the formation of impurity G in the high conversion DMAC/H₂O coupling reaction. Results, presented in Table 6 below, indicate that both KI and NaI suppress the formation of impurity G, with NaI appearing to have the greatest effect. Furthermore, it appears that the greater the equivalents of salt used, the lower the formation of impurity G in the coupling reaction.

TABLE 6

| | | | | | | | Over- | |
|---|---|---|---|---|---|---|---|---|
| | Base | | Solvent | Cmpd | | | alkylated | |
| Exp. | (eq.) | Epi. (eq.) | (mL/g) | A | Iohexol | Iodixanol | Imp. | Imp. G |
| 3954 × 100 | 0.65 | 0.31 | 3 | | | | | |
| 22.5 hr. | | Kl = 1 eq | | 25.23 | 16.17 | 53.75 | 2.01 | 0.28 |
| 46.5 hr. | | | | 13.44 | 13.34 | 65.72 | 2.81 | 0.64 |
| 72 hr. | | | | 10.21 | 15.80 | 68.12 | 3.15 | 0.88 |
| 95 hr. | | | | 9.19 | 16.78 | 68.50 | 3.26 | 0.98 |
| 167 hr. | | | | 7.92 | 17.33 | 68.84 | 3.34 | 1.34 |
| 3954 × 110 | 0.72 | 0.34 | 3 | | | | | |
| 22 hr. | | Nal = 0.25 | | 12.97 | 10.25 | 64.90 | 4.19 | 1.22 |
| | | eq | | | | | | |
| 22 hr. | | Nal = 0.50 | | 13.55 | 12.79 | 63.88 | 3.95 | 0.93 |
| | | eq | | | | | | |
| 22 hr. | | Nal = 0.75 | | 13.91 | 14.51 | 62.84 | 3.67 | 0.81 |
| | | eq | | | | | | |
| 22 hr. | | Nal = 1.0 | | 14.60 | 15.62 | 62.19 | 3.35 | 0.64 |
| | | eq | | | | | | |
| 22 hr. | | Nal = 1.25 | | 15.73 | 17.13 | 60.39 | 2.92 | 0.48 |
| | | eq | | | | | | |
| 22 hr. | | Nal = 1.50 | | 15.55 | 17.90 | 59.71 | 3.19 | 0.48 |
| | | eq | | | | | | |
| 47 hr. | | Nal = 0.25 | | 3.92 | 10.61 | 69.19 | 7.66 | 4.29 |
| | | eq | | | | | | |
| 47 hr. | | Nal = 0.50 | | 4.22 | 13.71 | 69.63 | 6.81 | 3.24 |
| | | eq | | | | | | |
| 47 hr. | | Nal = 0.75 | | 4.42 | 14.97 | 69.19 | 6.51 | 2.88 |
| | | eq | | | | | | |
| 47 hr. | | Nal = 1.0 | | 4.95 | 15.68 | 68.89 | 6.33 | 2.26 |
| | | eq | | | | | | |
| 47 hr. | | Nal = 1.25 | | 5.26 | 17.16 | 68.67 | 5.24 | 1.78 |
| | | eq | | | | | | |
| 47 hr. | | Nal = 1.50 | | 5.39 | 17.85 | 68.02 | 5.24 | 1.66 |
| | | eq | | | | | | |
| 144 hr. | | Nal = 0.25 | | 2.18 | 14.01 | 68.54 | 8.47 | 4.99 |
| | | eq | | | | | | |
| 144 hr. | | Nal = 0.50 | | 2.24 | 14.97 | 69.03 | 7.56 | 4.09 |
| | | eq | | | | | | |
| 144 hr. | | Nal = 0.75 | | 2.45 | 15.98 | 67.87 | 7.22 | 3.74 |
| | | eq | | | | | | |
| 144 hr. | | Nal = 1.0 | | 2.78 | 16.19 | 68.63 | 6.55 | 3.32 |
| | | eq | | | | | | |
| 144 hr. | | Nal = 1.25 | | 2.85 | 17.53 | 68.46 | 5.69 | 2.56 |
| | | eq | | | | | | |
| 144 hr. | | Nal = 1.50 | | 3.14 | 18.55 | 66.88 | 5.67 | 2.39 |
| | | eq | | | | | | |
| 3954 × 113 | 0.72 | 0.34 | 3 | | | | | |
| 23 hr. | | Nal = 1.0 | | 8.52 | 16.54 | 65.47 | 4.72 | 1.66 |
| | | eq | | | | | | |

### Example 7

### Compound A Coupling—High Conversion (H₂O) with KI

Reactions were run to see if adding KI could suppress the formation of impurity G in the coupling reaction with only H₂O as the solvent. Results, presented in Table 7 below, indicate that the addition of KI does suppress the formation of impurity G. However, the reactions carried out in water have been observed to already produce relatively small amounts of impurity G, and so the effect of KI addition here may be minimal.

TABLE 7

| | | | | | | | Over- | |
|---|---|---|---|---|---|---|---|---|
| | Base | | Solvent | Cmpd | | | alkylated | Imp. |
| Exp. | (eq.) | Epi. (eq.) | (mL/g) | A | Iohexol | Iodixanol | Imp. | G |
| 3954 × 101 | 0.65 | 0.31 | 3 | | | | | |
| 22.5 hr. | | Kl = 1 eq. | | 19.42 | 17.68 | 55.06 | 4.11 | 0.22 |
| 46.5 hr. | | | | 13.07 | 19.45 | 59.70 | 5.03 | 0.32 |
| 72 hr. | | | | 11.48 | 20.95 | 60.66 | 4.81 | 0.27 |
| 95 hr. | | | | 10.76 | 21.37 | 60.67 | 5.23 | 0.33 |

### Example 8

### Compound A Coupling—High Conversion (DMAc/H₂O)

A reaction was run to determine conversion of Compound A to iodixanol in a mixed solvent system of DMAc/H₂O without the addition of a metal iodide salt. The result are presented in Table 8, below.

TABLE 8

| | | | | | | | Over- | |
|---|---|---|---|---|---|---|---|---|
| | Base | | Solvent | Cmpd | | | alkylated | |
| Exp. | (eq.) | Epi. (eq.) | (mL/g) | A | Iohexol | Iodixanol | Imp. | Imp. G |
| 3954 × 107 | 0.621 | 0.544 | 2 | | | | | |
| 17 hr. | | | | 21.00 | 10.95 | 61.72 | 3.53 | 0.75 |
| 22 hr. | | | | 17.62 | 10.08 | 65.46 | 3.84 | 0.92 |
| 43.5 hr. | | | | 11.80 | 9.62 | 71.36 | 4.58 | 1.48 |
| 65.5 hr. | | | | 10.21 | 9.48 | 72.13 | 4.68 | 2.16 |

Although the present disclosure has been described with reference to preferred embodiments, persons skilled in the art will recognize that changes may be made in form and detail without departing from the spirit and scope of the disclosure.

When introducing elements of the present disclosure or the embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

1. The process for preparing a triiodinated X-ray contrast agent, the process comprising contacting in a reaction mixture a triiodo-substituted arylamide having Structure (I-A): 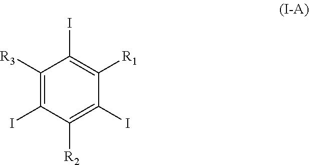
with an alkylating agent in the presence of a base, a suitable solvent, and an alkali metal iodide salt, wherein the molar ratio of the alkali metal iodide salt to the triiodo-substituted arylamide is at least about 1:1, and further wherein:
(i) R₁, R₂ and R₃ may be the same or different, and further may be independently selected from —NH—R₅, —C(O)—NH—R₆, or —NH—C(O)—R₆, provided at least one of R₁, R₂ and R₃ has one of the following structures: 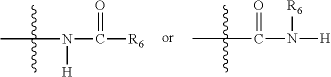
and,
(ii) R₅ and R₆ may be the same or different and may be independently selected from hydrogen, or substituted or unsubstituted alkyl, provided that R₆ is not hydrogen when R₁, R₂ or R₃ has the structure —NH—C(O)—R₆.

2. The process of claim 1 wherein at least one of R₁, R₂ and R₃ in the triiodo-substituted arylamide of Formula (I) has the structure: 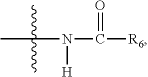
and further wherein R₆ is substituted or unsubstituted alkyl.

3. The process of claim 2 wherein only one of R₁, R₂ and R₃ has the structure, 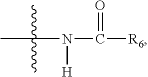
while the other two have the structure:

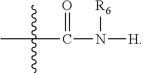

4. The process of claim 1 wherein a concentration of fused bicyclic impurities in the reaction mixture is less than about 5 area %, relative to the total concentration of the triiodinated X-ray contrast agent reaction product in the reaction mixture.

5. The process of claim 1 wherein the molar ratio of alkali metal iodide salt to triiodo-substituted arylamide of Structure (I-A) is between about 1:1 and about 2:1.

6. The process of claim 1 wherein the alkali metal iodide salt is selected from potassium iodide, sodium iodide, lithium iodide and cesium iodide.

7. The process of claim 6 wherein the alkali metal iodide salt is potassium iodide.

8. The process of claim 1 wherein the solvent is a mixed solvent system comprising a non-aqueous solvent and water, wherein the volume ratio of the non-aqueous solvent to water is greater than 1:1.

9. The process of claim 8 wherein the volume ratio of non-aqueous solvent to water is between greater than 1:1 and less than about 10:1.

10. The process of claim 8 wherein the alkylating agent is selected from the group consisting of 1,3-dichloro-2-propanol, 1-chloro-2,3-propane diol, 1-chloro-3-methoxy-2-propanol, and epichlorohydrin.

11. The process of claim 8 wherein the molar ratio of the triiodo-substituted arylamide compound and the alkylating compound is between about 2:1 and about 1:3.

12. The process of claim 1 wherein the amide compound is 5-acetamido-N,N-bis(2,3-dihydroxypropyl)-2,4,6-triiodoisophthalamide.

13. The process of claim 12 wherein the triiodinated X-ray contrast media reaction product is iodixanol.

14. The process of claim 1 wherein the triiodo-substituted arylamide of Structure (II-A) is contacted with an alkylating agent, LG-R₇—OH, a base, a solvent and an alkali metal iodide salt to form the triiodinated X-ray contrast agent of Structure (II-B): 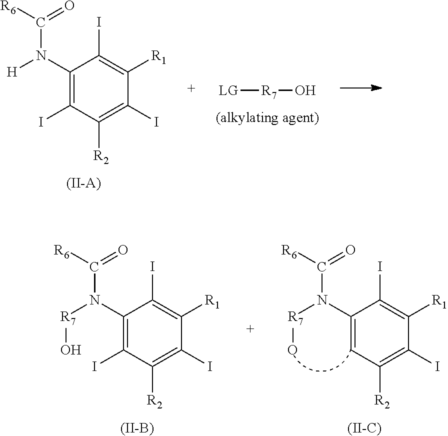
while limiting a concentration of a fused bicyclic impurity of Structure (II-C) in the reaction mixture to less than about 5 area %, relative to the total concentration of the triiodinated X-ray contrast agent reaction product in the reaction mixture, wherein:
(i) R₁, R₂, and R₆ are as defined in claim 1;
(ii) LG is a leaving group that is displaced during the reaction from the alkylating agent; and,
(iii) R₇—OH is a hydroxyl-substituted methyl, ethyl or propyl substituent, optionally substituted with one or more additional hydrocarbyl or heterohydrocarbyl substituents.

15. The process of claim 1 wherein about two equivalents of the triiodo-substituted arylamide of Structure (II-A) is contacted with about one equivalent of a dialkylating agent having the formula: 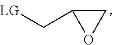
a base, a solvent and an alkali metal iodide salt to form the triiodinated X-ray contrast agent of Structure (II-DD): 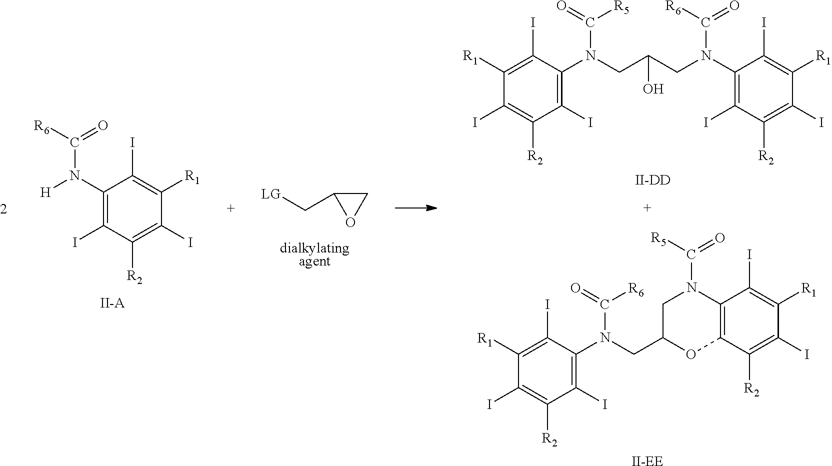
while limiting a concentration of a fused bicyclic impurity of Structure (II-EE) in the reaction mixture, wherein:
(i) R₁, R₂, and R₆ are as defined in claim 1; and,
(ii) LG is a leaving group that is displaced during the reaction from the alkylating agent.

16. The process of claim 15 wherein the triiodo-substituted arylamide is contacted with the dialkylating agent, the base, the solvent and the metal iodide salt to form the triiodinated X-ray contrast agent of Structure (II-DD), while limiting a concentration of a fused bicyclic impurity of Structure (II-EE) in the reaction mixture to less than about 5 area %, relative to the total concentration of the triiodinated X-ray contrast agent reaction product in the reaction mixture.

17. The process of claim 15 wherein about two equivalents of the triiodo-substituted arylamide of Structure (III-A) is contacted with about one equivalent of a dialkylating agent epichlorohydrin, a base, a solvent and an alkali metal iodide salt to form the triiodinated X-ray contrast agent of Structure (III-B): 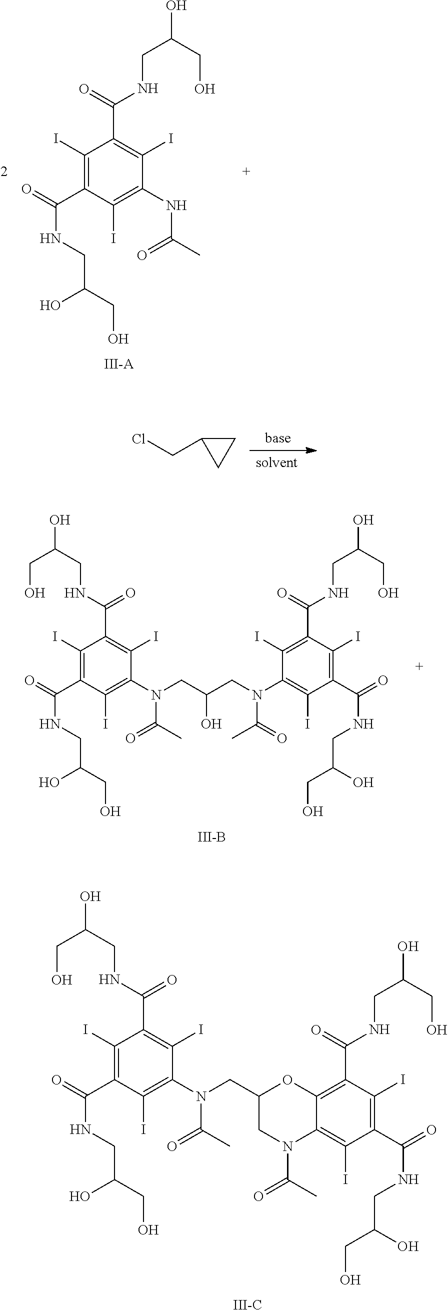
while limiting a concentration of a fused bicyclic impurity of Structure (III-C) in the reaction mixture.

18. The process of claim 17 wherein the triiodo-substituted arylamide is contacted with the dialkylating agent, the base, the solvent and the metal iodide salt to form the triiodinated X-ray contrast agent of Structure (III-B), while limiting a concentration of a fused bicyclic impurity of Structure (III-C) in the reaction mixture to less than about 5 area %, relative to the total concentration of the triiodinated X-ray contrast agent reaction product in the reaction mixture.

19. The process of claim 1 wherein the triiodo-substituted arylamide of Structure (IV-A) is contacted with an alkylating agent, LG-R₇—OH, a base, a solvent and an alkali metal iodide salt to form the triiodinated X-ray contrast agent of Structure (IV-B): 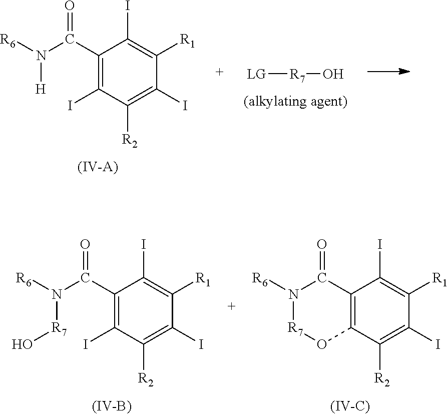
while limiting a concentration of a fused bicyclic impurity of Structure (IV-C) in the reaction mixture, wherein:
(i) R₁, R₂, and R₆ are as defined in claim 1;
(ii) LG is a leaving group that is displaced during the reaction from the alkylating agent; and,
R₇—OH is a hydroxyl-substituted methyl or ethyl substituent, optionally substituted with one or more additional hydrocarbyl or heterohydrocarbyl substituents.

20. The process of claim 19 wherein the triiodo-substituted arylamide is contacted with the dialkylating agent, the base, the solvent and the metal iodide salt to form the triiodinated X-ray contrast agent of Structure (IV-B), while limiting a concentration of a fused bicyclic impurity of Structure (IV-C) in the reaction mixture to less than about 5 area %, relative to the total concentration of the triiodinated X-ray contrast agent reaction product in the reaction mixture.
